(19) United States
(12) Patent Application Publication
(10) Pub. No.: US 2008/0305015 A1
(43) Pub. Date: Dec. 11, 2008

(54) Negative ion generator for incorporation into lighting apparatuses

(76) Inventors: Jason Ryu, Carlstadt, NJ (US); Chang-Min Lee, Seodaemun-Ku (KR)

Correspondence Address: RYNDAK & SURI LLP, 200 W. MADISON STREET, SUITE 2100, CHICAGO, IL, 60606, US

(21) Appl. No.: 12/221,908

(22) Filed: Aug. 7, 2008

Related U.S. Application Data
(63) Continuation-in-part of application No. 11/097,767, filed on Apr. 2, 2005.
(60) Provisional application No. 60/617,826, filed on Oct. 12, 2004.

(51) Int. Cl. A61L 9/00

(52) U.S. Cl. 422/122; 422/120

(57) ABSTRACT

An improved lighting and ionic air purifying device for both lighting and improving the air quality of a room or space. The device includes a light source such as a compact fluorescent lamp and an anion generator with a metal fiber brush which reduces or eliminates ozone production. The improved device provides extended lamp life and increased dispersion of ions into the ambient air in the room or space. The device may also include a photocatalyst coating on the lamp to further purify the air.

### RELATED APPLICATIONS

This application is a continuation-in-part of application Ser. No. 11/097,767, filed Apr. 2, 2005, which claims the benefit of U.S. Provisional Application No. 60/617,826, filed Oct. 12, 2004, the disclosures of which are hereby incorporated by reference in their entireties.

### FIELD OF THE INVENTION

The present invention relates to an ionizer apparatus for generating anions and purifying air, and more particularly to an improved ionizer apparatus incorporated into a lighting device for preventing generation of ozone while generating anions and purifying air, in addition to lighting a room.

### BACKGROUND OF THE INVENTION

Negative ions or anions have the effects of purifying blood, activating cells, increasing immunity, controlling the autonomic nervous system, removing toxic materials generated by electromagnetic waves, eliminating dust and bacteria, adjusting humidity and removing odor. When a person inhales such anions, there occur the beneficial effects of promoting the metabolism of cells, purifying blood, tranquilizing nerves, relieving fatigue and stimulating appetite. Under fresh natural conditions, it is known that the number of anions per 1 cc of air is 800-2,000, for example, in forests, near hot springs or waterfalls, and in seashore areas. Indoor air, on the other hand, often contains various contaminants such as dust, smoke, volatile organic compounds (VOCs) emitted by building materials and furnishings, bacteria, viruses, mold and the like. These contaminants tend to be positively charged as cations which deplete the density of anions in the air and lead to uncomfortable breathing and health risks. Anion generators can restore the balance of positive and negative ions, but conventional anion generators suffer from a number of problems. For example, they produce ozone and emit electromagnetic radiation, both of which can be harmful and are undesirable, and they require relatively high electric energy to operate.

A conventional anion generator includes an anion generating plate, a plurality of electron guns, an electronic plate having a plurality of holes and an anion collecting panel. The anion generator receives a high voltage from an amplifier and emits electrons to the electron guns mounted within the anion generator. The electrons discharged from the electron guns strike against the electron plate and the generate anions. The generated anions are discharged to the outside of the housing through the holes of the electronic plate. A problem with this type of anion generator is that it also produces ozone (O₃), which can be irritating in small quantities and lethal in larger concentrations. Other problems with conventional ion generators are that they generate electromagnetic radiation and require relatively high electric energy to operate. They also are often fairly large and inconvenient and they do not produce cluster ions which are the most desirable type.

Accordingly, it is desirable to provide an anion generator which produces beneficial anions but reduces or eliminates the generation of ozone, and may be conveniently incorporated into a lighting device such as a compact fluorescent light bulb (“CFL”). parent application Ser. No. 11/097,767 discloses a CFL with an ion generator. That device requires a wire to connect the metal fiber brush to the ion generator circuit. The wire not only adds cost to making the device, but also the electric field generated by the wire has been found to degrade the performance of the CFL and to shorten its lifespan. The position of the brush has been found to cause dust to build up on the portions of the bulb adjacent the brush. It is desirable to configure the device so that the performance and lifespan of the light bulb are not reduced. It is also desirable to improve airflow around the device to reduce dust accumulation on the CFL as well as to improve dispersion of ions into the ambient air.

### SUMMARY OF THE INVENTION

In a first embodiment of the invention there is provided an improved lighting apparatus for purifying the air without a UV lamp. The lighting apparatus uses a metal fiber brush which generates anions but reduces or eliminates the generation of ozone. The lighting apparatus also prevents anions generated from the anion generator from being removed by electromagnetic interference. The brush includes numerous metal fibers, each of the metal fibers being formed on an outer surface thereof with numerous tiny projections.

In another embodiment, an improved lighting apparatus includes an ion generator with a metal fiber brush which extends directly from the ion generator and is located in proximity to the housing of the apparatus.

In another aspect of the invention, apertures are provided in the housing to improve air flow around the device and to assist with dispersion of ions.

In another embodiment, a light bulb of the lighting apparatus may be coated with titanium dioxide, which acts as a photocatalyst when activated by light from the light bulb, to eliminate contaminants in the ambient air.

### BRIEF DESCRIPTION OF THE FIGURES

FIG. 1 is a partially cut-away perspective view showing a lighting device for generating anions and purifying air according to the present invention;

FIG. 2 is an exploded perspective view showing a lighting device for generating anions and purifying air according to the present invention;

FIG. 3 is a sectional view showing a lighting device for generating anions and purifying air according to the present invention; and

FIG. 4 is a circuit diagram of a lighting device for generating anions and purifying air according to the present invention.

### DETAILED DESCRIPTION OF THE INVENTION

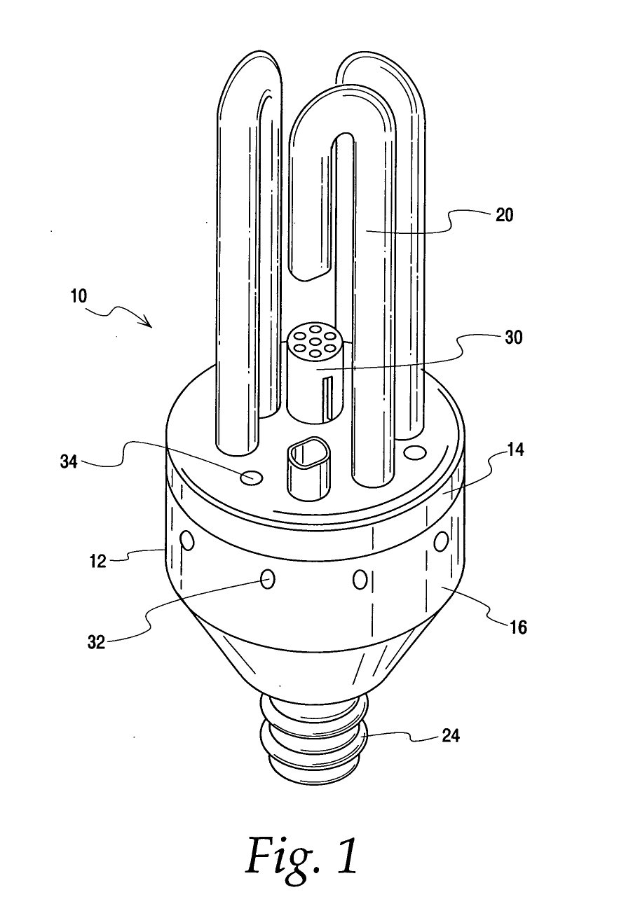

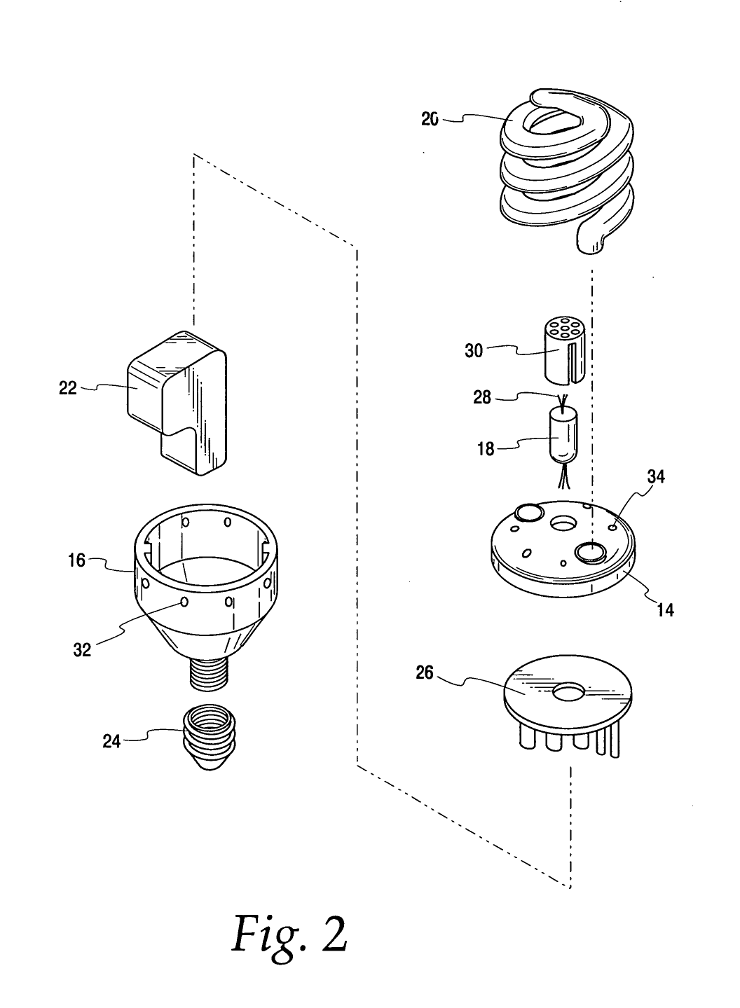

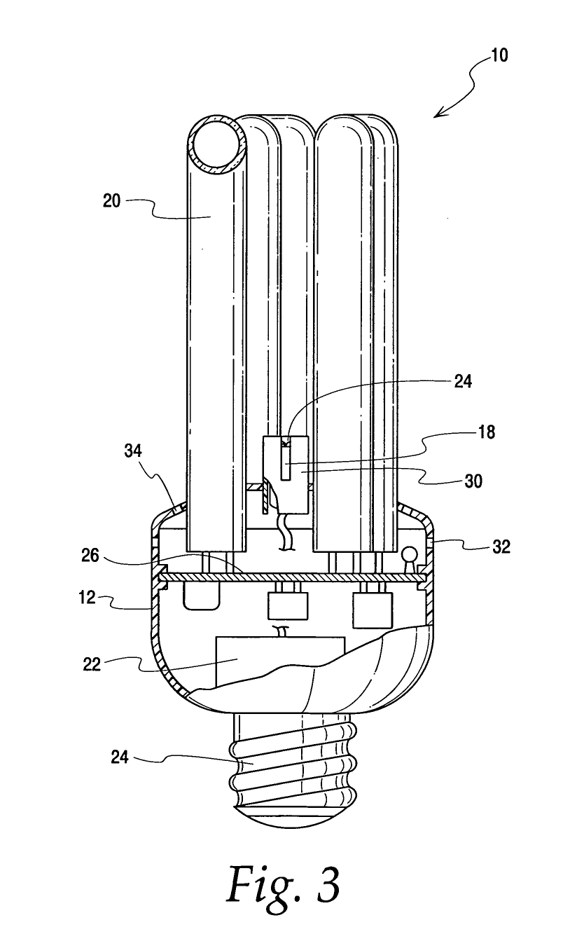

Referring to FIGS. 1 through 3, a lighting and air purifying apparatus 10 according to the present invention comprises a cylindrical housing 12 consisting of an upper part 14 and a lower part 16, an anion generator 18, a lamp 20 mounted on upper part 14 of housing 12; and an anion electric power source 22 engaged with a socket 24 within lower part 16 of housing 12. The anion electric power source 22 amplifies power to high voltages and applies the high voltages to the anion generator 18. Further, the electric power source 22 is fixedly mounted within housing 12. The anion electric power source 22 is preferably sealed within a case to prevent electromagnetic interference generated from the anion electric power source 22 from affecting a lamp electric power source 26 applying power to lamp 20. The invention is not limited to the spiral lamp configuration as illustrated in FIG. 2, and other types of lamps and/or a plurality of lamps are contemplated by the present invention. For example, FIGS. 1 and 3 illustrate a plurality of lamps 20 on housing 12.

Lamp electric power source 26 provides an appropriate voltage received from socket 24 to lamp 20. Lamp electric power source 26 includes a transformer and other related electric components, as in a conventional ballast unit for a fluorescent lamp. Lamp electric power source 26 is fixedly supported by members formed on an inner wall of housing 50.

Anion generator 18 is centrally mounted on upper part 14 of housing 12. Anion generator 18 includes a metal fiber (MF3) brush 28 for generating anions without generating ozone. Brush 28 has numerous tiny projections, and each of the projections has a sharp tip. Preferably, the metal fibers may be made of a conductive alloy, and more preferably the alloy is made up of eight different metals. Most preferably, the alloy comprises platinum, stainless steel, copper, silver, zinc, nickel, manganese and tungsten. The fibers may be bound together by rope-twisting them with a conductive resin and/or a semi conductive resin.

As shown in FIG. 2, brush 28 extends directly from anion generator 18 and thus is located just above the upper surface of upper part 14 of housing 12. This location has been found to reduce interference with lamp 20 as compared with devices in which the brush is located farther from housing 12 toward the center or far end of lamp 20. This arrangement also eliminates a wire extending from housing 12 to connect brush 28 to anion generator 18, the wire being a source of electromagnetic interference.

A non-conductive, perforated cap 30 may be provided to fit over brush 28 and anion generator 18 to protect brush 28 and prevent accidental contact by a user. The cap 30 may be, for example, of plastic and provided with holes, slots or other perforations to permit the anions to be discharged.

Apertures 32 and 34 are provided in the side and upper surfaces, respectively, of upper part 14 of housing 12. As a result of heat generated by anion power source 22 and lamp power source 26, air flows into apertures 32, through the interior of housing 12, and out though apertures 34. This serves to cool anion power source 22 and lamp power source 26, resulting in longer lifespans of these components. Further, the air flowing out through apertures 34 passes brush 28. Anions are discharged by brush 28 and moved by this airflow. The air flowing past brush 28 reacts with the generated anions and is converted into anions (i.e., the number of anions is increased). During the repeated performance of operation, numerous anions are generated in the air surrounding brush 28 and are dispersed into the ambient air. The airflow also carries away particles suspended in the air and tends to prevent the accumulation of dust on lamp 20 and housing 12. Apertures 32, 34 may be round holes as illustrated, or may be in the form of slots or other perforations.

As previously discussed, conventional anion generators using electronic guns generate ozone, which is undesirable, in addition to anions. In the lighting apparatus of the present invention, however, anion generator 18 comprises the metal fiber brush 28 generating anions without generating ozone. Thus, since the lighting apparatus of the present invention does not generate the air pollutant ozone and decomposes or dissolves organic substances in the air, it provides users with more purified air.

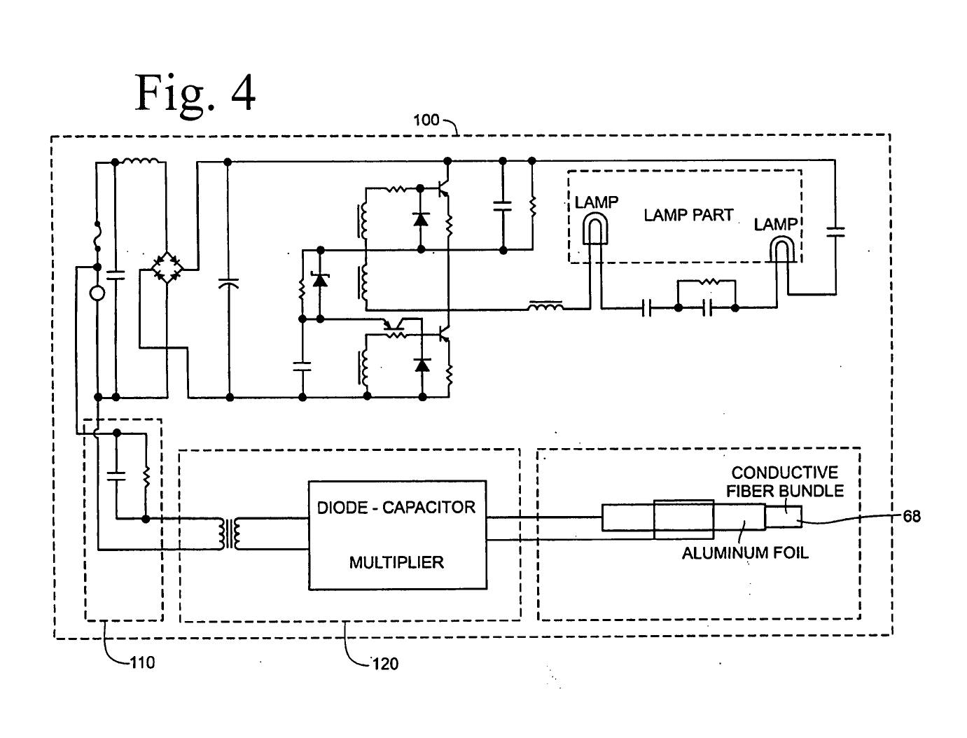

Referring to FIG. 4, the lighting apparatus of the present invention comprises a circuit 100 for receiving AC electric power, rectifying the AC electric power into DC electric power, applying the DC electric power to a ballast, and performing amplification of voltage and supply of constant voltage, thereby turning on the lamps.

Further, the circuit includes an interference protection unit 110 and an ion generation voltage induction unit 120 which provides high voltage to the metal fiber brush 28. The interference protection unit 110 receives AC electric power, and prevents the ballast 26 and the anion generator 18 from interfering with each other. The ion generation voltage induction unit 120 converts the AC electric power applied to the protection unit 110 into DC electric power of −5 kV, and generates pulses with a frequency multiplied by an integer ratio by a diode-capacitor multiplier. The metal fiber brush 28 receives current from the induction unit 120.

As is apparent in from the above description, the lighting apparatus of the present invention purifies the air without a UV lamp. Further, the lighting apparatus does not generate significant amounts of ozone because it uses the metal fiber brush. Further, the lighting apparatus prevents anions generated from the anion generator from being moved by electromagnetic interference.

Preferably, a titanium dioxide coating layer serving as a photocatalyst is applied to the exterior of lamp 20. The titanium dioxide is activated by light rays generated from the lamp 20, so that it decomposes or dissolves organic or inorganic substances. That is, when the substances approach to or come in contact with the photocatalyst activated by the light rays, they are subjected to oxidation and/or reduction reactions, so that they are decomposed or dissolved by a photocatalyst effect. The use of the titanium dioxide coating provides disinfection and deodorization of the air in addition to the generation of desirable anions in the same compact, convenient lighting device. In another embodiment, a plurality of light emitting diodes (“LEDs”) may replace the CFL. The LEDs may be monochromatic or polychromatic LEDs, as occasion demands, and the number of the LEDs may be varied and/or appropriately adjusted.

Although the preferred embodiments of the present invention have been disclosed for illustrative purposes, those skilled in the art will appreciate that various modifications, additions and substitutions are possible without departing from the scope and spirit of the invention as disclosed.

We claim:

1. A lighting and air purifying apparatus comprising:
a housing;
an anion generator mounted on the housing;
a metal fiber brush electrically connected to and extending directly from the anion generator;
an illumination unit mounted on the housing;
a power source mounted in the housing for supplying electric power to the ion generator; and
a power source mounted in the housing for supplying electric power to the illumination unit.

2. The apparatus of claim 1, wherein the metal fiber brush is made of a metal alloy consisting of platinum, stainless steel, copper, silver, zinc, nickel, manganese and tungsten.

3. The lighting apparatus of claim 1, wherein the metal fiber brush comprises a plurality of sharp protrusions.

4. The lighting apparatus of claim 1 wherein the illumination unit comprises a fluorescent tube.

5. The lighting apparatus of claim 4, wherein the fluorescent tube is coated with a photocatalyst.

6. The lighting apparatus of claim 5, wherein the photocatalyst is titanium dioxide.

7. The lighting apparatus of claim 1 wherein the illumination unit comprises at least one light emitting diode.

8. The apparatus of claim 1 wherein the housing has an upper surface and a side surface, the upper surface and side surface each having at least one aperture therein for permitting air to flow through the housing.

9. The apparatus of claim 8 wherein the apertures are disposed such that ambient air enters the at least one aperture in the side surface, flows through the interior of the housing, flows out of the housing through the at least one aperture in the upper surface and flows past the metal fiber brush so as to disperse ions generated by the brush.
